(19) United States
(12) Patent Application Publication
(10) Pub. No.: US 2018/0328657 A1
(43) Pub. Date: Nov. 15, 2018

(54) METHODS FOR PRODUCING PRESSURIZED ALKENE GAS

(71) Applicant: Linde Aktiengesellschaft, Munich (DE)

(72) Inventor: Nicole RUMORE, Monmouth Junction, NJ (US)

(21) Appl. No.: 15/591,869

(22) Filed: May 10, 2017

(51) Int. Cl. F25J 3/06; C07C 7/09

(52) U.S. Cl. CPC F25J 3/064; C07C 7/09; F25J 2215/64; F25J 2215/04; F25J 2270/90; F25J 2280/20; F25J 2280/02; F25J 2215/62

(57) ABSTRACT

Methods and systems for recovering alkene (e.g., C₂-C₄ alkene) gas as well as producing pressurized alkene (e.g., C₂-C₄ alkene) gas from process gas streams including lower concentrations of alkenes are provided herein.

### FIELD OF THE INVENTION

This invention relates to methods for recovering alkene gas, such as ethylene gas and propylene gas from various process gas streams as well as methods for producing pressurized alkene gas streams.

### BACKGROUND OF THE INVENTION

Valuable hydrocarbons, such as lower alkenes, may be present in a multitude of process gas streams at varying concentrations and typically amongst other components. For example, ethylene and/or propylene may be present in cracked gas streams from hydrocarbon crackers, which also may contain nitrogen, methane, ethane, propane, hydrogen, and other higher hydrocarbons. Additionally, during polymerization processes, unreacted ethylene or propylene monomer may be present in streams along with inert purge gases, such as nitrogen and/or argon. For example, during production of polyethylene, a large quantity of nitrogen and/or argon may be used for blanketing, which can reduce the flammability risk of the hydrocarbons within the process. Typically, streams containing alkene process gas (e.g., ethylene, propylene) are removed from the system via flaring or with a thermal oxidizer. Prior to flaring or thermal oxidizing, recovery processes may be employed for recovering as much as of the alkene as possible so it is not lost when flared or oxidized. Examples of existing recovery processes include compression and condensation systems as well as use of pressure swing adsorption systems and/or membranes. However, existing recovery processes may be limited in the amount of alkene recovery, especially where alkene concentration is low, and even further processing may be required at additional costs to recover further amounts of alkene,

Additionally, such process gas streams are typically at a lower pressure. However, recovered alkenes may require high pressure to be transported and utilized throughout various systems. Pressurizing the recovered alkenes can be accomplished through use of a compressor or cryogenic pump (e.g., for liquid alkenes), but such methods may be costly due to required maintenance and/or high power consumption. Thus, a need remains for improved methods for recovery of alkene gas from process gas streams as well as improved methods for pressurized alkene gas, such as pressurized ethylene or pressurized propylene.

### SUMMARY OF THE INVENTION

It has been found that a pressurized alkene gas, such as pressurized ethylene or pressurized propylene can be achieved by performing a combination of process steps including condensing a process gas stream comprising alkenes followed by pressurizing the resultant condensate via heating.

Thus, in one aspect, this disclosure relates to a method for producing pressurized C₂-C₄ alkene gas from a process gas stream comprising: a condensation cycle comprising: cooling the process gas stream comprising less than 80 wt % C₂-C₄ alkene in at least one condenser vessel with a cooling medium under suitable conditions to produce a first condensate comprising C₂-C₄ alkene and a vent gas stream, wherein the first condensate is collected in at least one condensate tank; and a pressurization cycle comprising: halting flow of the process gas stream and the cooling medium to the at least one condenser vessel and flow of the first condensate stream to the at least one condensate tank when the first condensate reaches a first high level in the at least one condensate tank; and heating the at least one condensate tank to produce pressurized liquid C₂-C₄ alkene and a C₂-C₄ alkene vent gas

In still another aspect, this disclosure relates to a system for producing a pressurized C₂-C₄ alkene gas from a process gas stream comprising: a process gas stream comprising less than 80 wt % alkene; a cooling medium stream; a vent gas stream; a first condensate stream comprising C₂-C₄ alkene; a pressurized liquid C₂-C₄ alkene stream; a C₂-C₄ alkene vent gas stream; a pressurized C₂-C₄ alkene gas stream; at least one condenser vessel operated under suitable conditions to produce the first condensate stream comprising C₂-C₄ alkene and the vent gas stream, wherein the at least one condenser vessel comprises: a shell and tube heat exchanger or a plate heat exchanger; a first inlet for providing the process gas stream; a second inlet for providing the cooling medium; a first outlet for removal of a spent cooling medium; a second outlet for removal of the vent gas stream; and a third outlet for removal of the first condensate; at least one condensate tank, wherein the at least one condensate tank comprises: a third inlet for providing the first condensate stream; a fourth outlet for removal of the pressurized liquid C₂-C₄ alkene stream; and a fifth outlet for removal the C₂-C₄ alkene vent gas stream; a means for providing heat to the at least one condensate tank; and at least one vaporizer operated under suitable conditions to produce the pressurized C₂-C₄ alkene gas stream having a pressure of at least about 100 kPa and a temperature higher than a temperature of the pressurized liquid C₂-C₄ alkene, wherein the vaporizer comprises: a fourth inlet for providing the pressurized liquid C₂-C₄ alkene stream; and a sixth outlet for removal of the pressurized C₂-C₄ alkene gas stream.

Other embodiments, including particular aspects of the embodiments summarized above, will be evident from the detailed description that follows.

### BRIEF DESCRIPTION OF THE DRAWINGS

The drawings described herein are for illustrative purposes only of selected embodiments and not all possible implementations, and are not intended to limit the scope of the present disclosure.

FIG. 1 illustrates a sequence of cycles in two systems running in parallel for producing pressurized alkene gas.

FIG. 2 illustrates a schematic of a system for producing pressured alkene gas according to certain aspects of the present disclosure.

FIG. 3 illustrates a schematic of a system for producing pressurized alkene gas according to certain alternative aspects of the present disclosure.

Corresponding reference numerals indicate corresponding parts throughout the several views of the drawings.

### DETAILED DESCRIPTION OF THE INVENTION

In various aspects of the invention, methods and systems for recovering alkene gas and producing pressurized alkene gas from process gas streams are provided.

### I. Definitions

The term “and/or” as used in a phrase such as “A and/or B” herein is intended to include “A and B”, “A or B”, “A”, and “B”.

As used herein, and unless otherwise specified, the term “Cₙ” means hydrocarbon(s) having n carbon atom(s) per molecule, wherein n is a positive integer. As used herein, and unless otherwise specified, the term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different values of n.

As used herein, the term “alkene,” alternatively referred to as “olefin,” refers to a branched or unbranched unsaturated hydrocarbon having one or more carbon-carbon double bonds. A simple alkene comprises the general formula CₙH₂ₙ, where n is 2 or greater. Examples of alkenes include, but are not limited to ethene, propene, butene, pentene, hexene and heptene. “Alkene” is intended to embrace all structural isomeric forms of an alkene. For example, butene encompasses but-1-ene, (Z)-but-2-ene, etc.

### II. Methods for Producing Pressurized Alkene Gas

Methods for producing pressurized alkene gas are provided herein. The methods may comprise a condensation cycle and a pressurization cycle.

A. Condensation Cycle

During the condensation cycle, a process gas stream may be cooled in at least one condenser vessel with a cooling medium under suitable conditions to produce a first condensate and a vent gas stream. The process gas stream comprises alkene gas, e.g., C₂-C₁₀ alkenes, C₂-C₈ alkenes or C₂-C₄ alkenes. In particular the process gas stream comprises ethylene and/or propylene. Additionally, the process gas stream may comprise alkene gas (e.g., C₂-C₄ alkenes), in an amount, based on the total weight of the process gas stream, of less than or equal to about 85 wt %, less than or equal to about 80 wt %, less than or equal to about 75 wt %, less than or equal to about 70 wt %, less than or equal to about 65 wt %, less than or equal to about 60 wt %, less than or equal to about 55 wt %, less than or equal to about 50 wt %, less than or equal to about 45 wt %, less than or equal to about 40 wt %, less than or equal to about 35 wt %, less than or equal to about 30 wt %, less than or equal to about 25 wt %, less than or equal to about 20 wt %, less than or equal to about 15 wt %, less than or equal to about 10 wt %, or about 1.0 wt %. In particular, the process gas stream may comprise alkene gas (e.g., C₂-C₄ alkenes), singularly or in combination, in an amount, based on the total weight of the process gas stream, of less than or equal to 80 wt %. It is understood herein that the amount of alkene gas provided herein corresponds to both a single alkene amount as well as combined amounts of alkenes, if one or more are present. For example, an alkene gas (e.g , C₂-C₄ alkenes) present in an amount of less than or equal to 80 wt % encompasses a process stream comprising less than or equal to 80 wt % ethylene as well as a process stream comprising less than or equal to 80 wt % ethylene and propylene in combination. Additionally or alternatively, the process gas stream may comprise alkene gas (e.g., C₂-C₄ alkenes) in an amount, based on the total weight of the process gas stream, of about 1.0 wt % to about 85 wt %, about 1.0 wt % to about 80 wt %, about 1.0 wt % to less than about 80 wt %, about 1.0 wt % to about 60 wt % or about 10 wt % to about 50 wt %.

A remaining portion, e.g., the balance, of the process gas stream may further comprise impurities, such as, but not limited to methane, ethane, propane, and hydrogen, and other components, such as nitrogen. For example, the impurities (e.g., methane) and/or other components (e.g., nitrogen) may be present in the process gas stream in amount of at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt or about 99 wt %. In particular, the impurities (e.g., methane) and/or other components (e.g., nitrogen) may be present in the process gas stream in amount of at least about 20 wt %. Additionally or alternatively, the impurities (e.g., methane) and/or other components (e.g., nitrogen) may be present in the process gas stream in amount of about 15 wt % to about 99 wt %, about 20 wt % to about 99 wt %, about 20 wt % to about 90 wt %, about 30 wt % to about 80 wt %, or about 40 wt % to about 75 wt %.

In various aspects, the process gas stream may enter the at least one condenser vessel at any suitable temperature and/or pressure, for example, as determined by previous process steps and conditions for producing the process gas stream. For example, the process gas stream may enter the at least one condenser vessel at a temperature of about 0.0° C. or lower, about −10° C. or lower, about −20° C. or lower, about −30° C. or lower, about −40° C. or lower, about −50° C. or lower, about −60° C. or lower, about −70° C. or lower, about −80° C. or lower, about −90° C. or lower or about −100° C. Additionally or alternatively, the process gas stream may enter the at least one condenser vessel at a temperature of about −100° C. to about 0.0° C., about −100° C. to about −10° C., about −100° C. to about −20° C., about −100° C. to about −30° C., about −100° C. to about −40° C., about −80° C. to about −0.0° C., about −80° C. to about −10° C., about −80° C. to about −20° C., about −80° C. to about −30° C., or about −80° C. to about −40° C. Additionally, the process gas stream may enter the at least one condenser vessel at a pressure, optionally in combination with the above-described temperatures, of at least about 80 kPa, at least about 90 kPa, at least about 100 kPa, at least about 110 kPa, at least about 120 kPa, at least about 150 kPa, at least about 180 kPa, at least about 200 kPa, at least about 220 kPa, at least about 250 kPa, at least about 280 kPa, or about 300 kPa. For example, the process gas stream may enter the at least one condenser vessel at a pressure, optionally in combination with the above-described temperatures, of about 80 kPa to about 300 kPa, about 90 kPa to about 250 kPa, about 90 kPa to about 200 kPa, about 90 kPa to about 150 kPa or about 90 kPa to about 110 kPa. In particular, the process gas stream may enter the at least one condenser vessel at a temperature of −40° C. or lower and a pressure of at least about 90 kPa.

Further, during the process, the pressure in the at least one condenser vessel may be maintained at about 90 kPa to about 500 kPa, about 100 kPa to about 400 kPa, or about 100 kPa to about 300 kPa. In particular, the pressure in the at least one condenser vessel may be maintained at about 100 kPa to about 300 kPa. When the pressure in the condenser vessel falls below a predetermined lower limit, for example, lower than 100 kPa or 90 kPa, as the alkene gas condenses, the flow of process gas into the at least one condenser vessel may be increased to increase pressure within the condenser vessel.

In order to produce the first condensate, a cooling medium may be circulated through the at least one condenser vessel, for example, via any suitable cooling means (e.g., within the condenser vessel, at a temperature suitable for condensing at least a portion of the alkene gas (e.g., C₂-C₄ alkenes) present in the process gas stream to produce the first condensate comprising alkenes (e.g., C₂-C₄ alkenes). A suitable cooling medium includes, but is not limited to liquid and/or gaseous nitrogen. The cooling medium (e.g., liquid and/or gaseous nitrogen) may be provided and/or circulated in the cooling means at a temperature of at least about −210° C., at least about −196° C., at least about −190° C., at least about −180° C., at least about −170° C., at least about −160° C., or at least about −150° C. For example, the cooling medium (e.g., liquid and/or gaseous nitrogen) may be provided and/or circulated in the cooling means at a temperature of about −196° C. to about −150° C., about −190° C. to about −150° C., or about −180° C. to about −160° C. Additionally, the cooling medium (e.g., liquid and/or gaseous nitrogen) may be provided and/or circulated in the cooling means, optionally in combination with the above-described temperatures, at a pressure of less than or equal to about 1600 kPa, less than or equal to about 1500 kPa, less than or equal to about 1300 kPa, less than or equal to about 1200 kPa, less than or equal to about 1000 kPa, less than or equal to about 800 kPa, less than or equal to about 700 kPa, less than or equal to about 500 kPa, less than or equal to about 300 kPa, or about 100 kPa. For example, the cooling medium (e.g., liquid and/or gaseous nitrogen) may be provided and/or circulated in the cooling means, optionally in combination with the above-described temperatures, at a pressure of about 100 kPa to about 1600 kPa, about 300 kPa to about 1500 kPa, about 500 kPa to about 1500 kPa, about 700 kPa to about 1300 kPa or about 800 kPa to about 1200 kPa. In particular, the cooling medium (e.g., liquid and/or gaseous nitrogen) may be provided and/or circulated in the cooling means at a temperature of at least about −196° C. and/or at a pressure of less than or equal to about 1500 kPa (e.g., about −170° C. and about 1000 kPa. Additionally or alternatively, the first condensate collecting in the at least one condenser vessel may have a temperature of about about −170° C. to about −104° C., about −170° C. to about −120° C., or about −170° C. to about −140° C., or about −150° C. to about −104° C. Examples of suitable cooling means include, but are not limited to a coil and heat exchangers, such as a shell and tube heat exchanger and a plate heat exchanger. In particular, the cooling means may be a shell and tube heat exchanger and a plate heat exchanger.

During the condensation cycle, the first condensate may drain out of the at least one condenser vessel and collect in at least one condensate tank. Further, a vent gas may be produced during the condensation cycle, and the vent gas may be removed from the condenser vessel. The vent gas may comprise the non-condensable components of the process gas stream. For example, vent gas may primarily comprise (e.g., ≥about 90 wt %, ≥about 95 wt %, ≥about 98 wt %, ≥about 99 wt %, or about 99.5 wt %) the impurities as described herein present in the process gas stream, such as but not limited to hydrogen, and/or methane, as well as other components, such as nitrogen. In particular, the vent gas comprises nitrogen. Additionally or alternatively, the vent gas may comprise trace amounts (e.g., ≤about 5.0 wt %, ≤about 2.0 wt %) of alkenes (e.g., C₂-C₄ alkenes).

In various aspects, a lower pressure drop may be maintained within the at least one condenser vessel. For example, in the at least one condenser vessel, the pressure drop may be less than about 20 kPa, less than about 15 kPa, less than about 10 kPa, less than about 7 kPa or about 5.0 kPa. In particular, in the at least one condenser vessel, the pressure drop may be less than about 10 kPa. Frozen particles (e.g., frozen alkene) may accumulate within the at least one condenser vessel, for example, on the surface (e.g., plates) of a heat exchanger, the pressure drop within the at least one condenser vessel may increase. In certain aspects, the condensation cycle may be halted once the pressure drop in the at least one condenser vessel reaches a predetermined value as necessitated by the process conditions, for example, a pressure drop greater than about 10 kPa, greater than about 12 kPa or greater than about 15 kPa.

In various aspects, the condensation cycle may run for about 1.0 hour to about 72 hours, about 3.0 hours to about 48 hours, about 6.0 hours to about 24 hours or about 6.0 hours to about 18 hours. In particular, the condensation cycle may run for about 6 hours to about 24 hours or about 8 hours to about 12 hours.

B. Pressurization Cycle

The pressurization cycle may comprise halting flow of the process gas stream and the cooling medium to the at least one condenser vessel and halting flow of the first condensate stream to the at least one condensate tank when the first condensate reaches a first high level in the at least one condensate tank and/or when pressure drop in the at least one condenser vessel exceeds a predetermined value as described herein. Optionally, the process gas stream may be directed to another condenser vessel in series where it may undergo a condensation cycle. The first high level in the at least one condensate tank may be any suitable level as determined by the process conditions. For example, the first high level in the at least one condensate tank may be when the first condensate is present in at least about 30%, at least about 40%, at least about 50%, at least about 60% at least about 70%, at least about 80%, or up to about 90% of the volume of the at least one condensate tank. Additionally or alternatively, the first high level in the at least one condensate tank may be when the first condensate is present at about 30% to about 90% of the volume of the at least one condensate tank, about 40% to about 90% of the volume of the at least one condensate tank, about 60% to about 90% of the volume of the at least one condensate tank, or about 70% to about 90% of the volume of the at least one condensate tank.

Additionally, the pressurization cycle may comprise heating the at least one condensate tank to produce pressurized liquid alkene (e.g., C₂-C₄ alkene) and an alkene (e.g., C₂-C₄ alkene) vent gas. The heat provided to the at least one condensate tank may vaporize at least a portion of the alkenes (e.g., C₂-C₄ alkene) in the first condensate to produce pressurized liquid alkene (e.g., C₂-C₄ alkene) and an alkene (e.g., C₂-C₄ alkene) vent gas. The alkene (e.g., C₂-C₄ alkene) vent gas may also comprise other components, such as methane. Any suitable means for providing heat to the condensate tank may be used, for example, heat may be provided via a heater (e.g., electric heater), via heated gaseous nitrogen, via heated air or via an ambient vaporizer. Further, the heat may be provided at suitable temperature for a suitable amount of time to produce pressurized liquid alkene (e.g., C₂-C₄ alkene) at a desirable pressure as determined by the needs of the process, for example, at a pressure of about 100 kPa to about 1500 kPa, about 200 kPa to about 800 kPa or about 300 kPa to about 500 kPa. Additionally or alternatively, the pressurized liquid alkene (e.g., C₂-C₄ alkene) may have a temperature of less than about −104° C., e.g., about −170° C. to about −104° C., about −170° C. to about −120° C., about −170° C. to about −140° C., or about −150° C. to about −104° C.

In various aspects, the pressurization cycle may further comprise passing the pressurized liquid alkene (e.g., C₂-C₄ alkene) through a vaporizer operated under suitable conditions to produce pressurized alkene (e.g., C₂-C₄ alkene) gas. Any suitable vaporizer, such as, but not limited to an ambient vaporizer or an electric vaporizer, may be used. The pressurized liquid alkene (e.g., C₂-C₄ alkene) may be passed through the vaporizer as determined by desired process conditions. For example, the pressurized liquid alkene (e.g., C₂-C₄ alkene) may be passed through the vaporizer when the pressure in the headspace of the at least one condensate tank reaches a predetermined value, e.g., a pressure of about 200 kPa to about 1500 kPa, about 200 kPa to about 800 kPa, or about 300 kPa to about 500 kPa.

Advantageously, the pressurized alkene (e.g., C₂-C₄ alkene) gas may have a temperature higher than a temperature of the pressurized liquid alkene (e.g., C₂-C₄ alkene) and/or a pressure of at least about 100 kPa, at least about 200 kPa, at least about 300 kPa, at least about 400 kPa, at least about 500 kPa, at least about 600 kPa, at least about 700 kPa, at least about 800 kPa, at least about 900 kPa, at least about 1000 kPa, at least about 1100 kPa, at least about 1200 kPa, at least about 1300 kPa, at least about 1400 kPa, at least about 1500 kPa or about 2000 kPa. Additionally or alternatively, the pressurized alkene (e.g., C₂-C₄ alkene) gas may have a pressure of about 100 kPa to about 2000 kPa, about 100 kPa to about 1500 kPa, or about 300 kPa to about 1500 kPa.

Passing of the pressurized liquid alkene (e.g., C₂-C₄ alkene) through a vaporizer may continue for as long as determined by desired process conditions. For example, passing of the pressurized liquid alkene (e.g., C₂-C₄ alkene) through the vaporizer may be halted when the pressurized liquid alkene (e.g., C₂-C₄ alkene) reaches a first low level in the at least one condensate tank. The first low level in the at least one condensate tank may be any suitable level as determined by the process conditions. For example, the first low level in the at least one condensate tank may be when the pressurized liquid alkene (e.g., C₂-C₄ alkene) is present in at most about 30%, at most about 20%, at most about 10%, or about 5.0% of the volume of the least one condensate tank. Additionally or alternatively, the first low level in the at least one condensate tank may be when the pressurized liquid alkene (e.g., C₂-C₄ alkene) is present at about 5.0% to about 30% of the volume of the at least one condensate tank, about 10% to about 30% of the volume of the at least one condensate tank, or about 20% to about 30% of the volume of the at least one condensate tank.

In various aspects, at least a portion of the alkene (e.g., C₂-C₄ alkene) vent gas may be removed from the condensate tank when desired, for example, to depressurize and/or empty the condensate tank. Further, at least a portion of the alkene (e.g., C₂-C₄ alkene) vent gas may be introduced into the at least one condenser vessel to recondense the alkenes and/or at least a portion of the alkene (e.g., C₂-C₄ alkene) vent gas may be flared or sent to a thermal oxidizer.

In various aspects, the pressurization cycle may run for about 1.0 hour to about 72 hours, about 3.0 hours to about 48 hours, about 6.0 hours to about 24 hours or about 6.0 hours to about 18 hours. In particular, the pressurization cycle may run for about 6 hours to about 24 hours or about 8 hours to about 12 hours.

C. Defrost Cycle

The methods described herein may further comprise a defrost cycle to melt any frozen alkenes and/or impurities in the at least one condenser vessel. The defrost cycle may be performed as needed by the process. For example, the defrost cycle may be commenced when the pressure drop within the condenser vessel increases to an undesirable level as described herein and/or when the level of condensate in the tank is too high. The defrost cycle may comprise halting the process gas stream and the cooling medium to the at least one condenser vessel and heating the at least one condenser vessel to produce a second condensate stream comprising alkenes (e.g., C₂-C₄ alkene). The heating of the at least one condenser vessel may be provided by any suitable means for defrosting the condenser vessel. For example, heating may be provided by gaseous nitrogen. The gaseous nitrogen may be heated to a suitable temperature (e.g., greater than about −104° C. up to about 25° C.) prior to introduction into the condenser vessel. The defrost cycle may further comprise draining the second condensate stream from the at least one condenser vessel to collect in a second condensate tank.

In various aspects, the defrost cycle may run for about 1.0 hour to about 18 hours, about 1.0 hour to about 12 hours, about 1.0 hour to about 6.0 hours, about 1.0 hour to about 3.0 hours, or about 1.0 hour to about 2.0 hours. In particular, the defrost cycle may run for about 1.0 hour to about 3.0 hours.

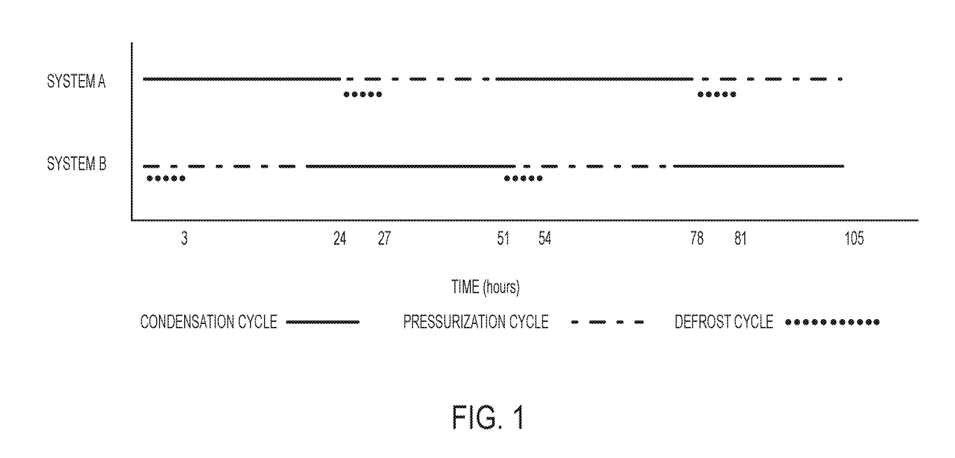

It is further contemplated herein, that the condensation cycle, the pressurization cycle, and the defrost cycle may run in parallel in two or more condenser vessels and associated condensate tanks (e.g., a third condensate tank, a fourth condensate tank, etc.), which may be in series. For example, as illustrated in FIG. 1, two systems (System A and System B) running in parallel may undergo a sequence of respective cycles as described herein. In particular, System A, which comprises at least one condenser vessel and an associated condensate tank, may be undergoing a condensation cycle followed by a defrost cycle and a pressurization cycle while System B, which comprises at least one condenser vessel and an associated condensate tank, may be undergoing a defrost cycle and a pressurization cycle followed by a condensation cycle and so on. As shown in FIG. 1, at least a portion of a defrost cycle and at least a portion of a pressurization cycle may optionally run simultaneously. For example, in System A, while the condenser vessel may be undergoing a defrost cycle, the associated condensate tank may undergo a pressurization cycle.

### III. Systems for Producing Pressurized Alkene Gas

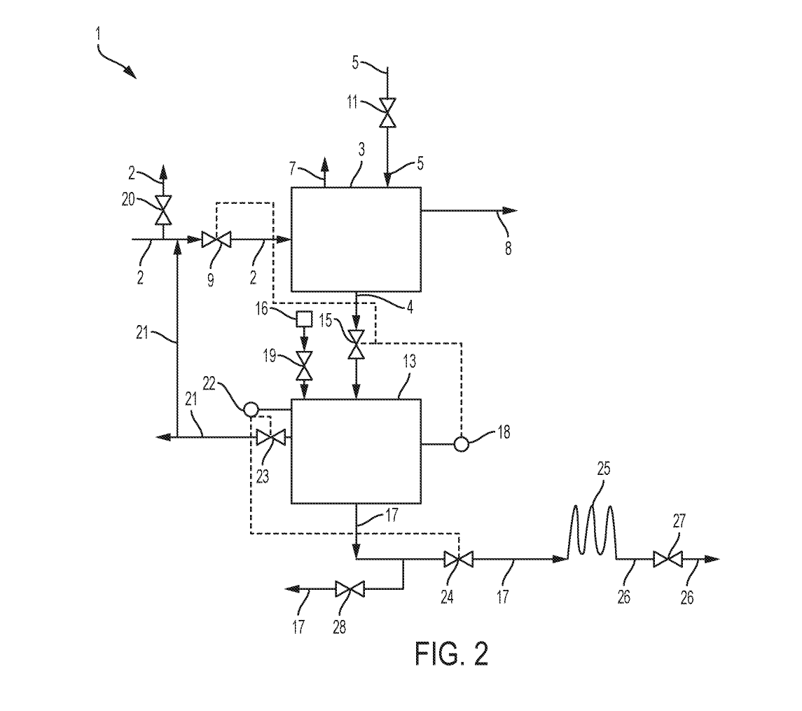

Systems for producing a pressured alkene gas as described herein are also provided. Referring to FIG. 2, the system 1 may comprise a process gas stream 2 as described herein, which is provided to at least one condenser vessel 3 via a first inlet (not shown), for example, during a condensation cycle as described herein. In particular, the process gas stream 2 may comprise alkene (e.g., C₂-C₄ alkenes) gas, singularly or in combination, in an amount, based on the total weight of the process gas stream, of less than 80 wt %. In certain aspects, the alkenes are ethylene and/or propylene. A valve 9 may control the flow of process gas stream 2 into the condenser vessel 3 (e.g., shell and tube heat exchanger, plate heat exchanger, coil heat exchanger).

Additionally, in order to produce a first condensate stream 4 as described herein, the system may further comprise a cooling medium stream 5 provided via a second inlet (not shown) and controlled by a valve 11, which may be circulated through the at least one condenser vessel 3 (e.g., shell and tube heat exchanger, plate heat exchanger) at a temperature suitable for condensing at least a portion of the alkene gas (e.g., C₂-C₄ alkenes) present in the process gas stream 2 to produce the first condensate stream 4 comprising alkenes (e.g., C₂-C₄ alkenes). The cooling medium stream 5 may comprise a suitable cooling medium as described herein, for example, liquid and/or gaseous nitrogen. In particular, the cooling medium stream 5 (e.g., liquid and/or gaseous nitrogen) may be provided and/or circulated in the condenser vessel 3 at a temperature of at least about −196° C. and/or at a pressure less than or equal to about 1500 kPa. As the cooling medium stream 5 (e.g., liquid and/or gaseous nitrogen) circulates through the condenser vessel 3, it may be heated and exit the system 1 as a spent cooling medium stream 7 via a first outlet (not shown). In various aspects, at least a portion of the spent cooling medium stream 7 may comprise gaseous nitrogen.

The system 1 may further comprise a vent gas stream 8 as described herein, which may be produced in the condenser vessel 3 during the condensation cycle. The vent gas may primarily comprise (e.g., ≥about 90 wt %, ≥about 95 wt %, ≥about 98 wt %, %, ≥about 99 wt %, or about 99.5 wt %) the non-condensable components of the process gas stream 2, e.g., hydrogen, nitrogen and/or methane. In particular, the vent gas stream 8 comprises nitrogen. As describe herein, the vent gas stream 8 may be removed from the condenser vessel 3 via a second outlet (not shown). For example, the vent gas stream 8 may be continuously removed from the condenser vessel 3.

At least one condensate tank 13 may be present in the system 1 for collection of the first condensate stream 4, which may be removed via a third outlet (not shown) in the condenser vessel 3, for example, as the first condensate stream 4 drains from the at least one condenser vessel 3 during the condensation cycle as described herein. The at least one condensate tank 13 may comprise a third inlet (not shown) for providing the first condensate stream 4.

The system may further comprise a means for providing heat 16 and valve 19 for providing heat to the condensate tank 13 to produce a pressurized liquid alkene (e.g., C₂-C₄ alkene) stream 17, for example, during a pressurization cycle as described herein. The means for providing heat 16 may be a heater as described herein, an ambient vaporizer, heated air, or heated gaseous nitrogen. The flow of the process gas stream 2 and the cooling medium stream 5 may be halted to the at least one condenser vessel 3 by closing valves 9 and 11 when the first condensate stream 4 reaches a first high level in the at least one condensate tank 13 as monitored by sensor 18. Optionally, the process gas stream 2 may be directed to another condenser vessel (not shown) via valve 20. Further a valve 15 may be closed to stop the flow of first condensate stream 4 to the at least one condensate tank 13. Then valve 19 may be opened to provide heat to the condensate tank 13 to produce the pressurized liquid alkene (e.g., C₂-C₄ alkene) stream 17 as well as an alkene (e.g., C₂-C₄ alkene) vent gas stream 21. The condensate tank 13 may further comprise a fourth outlet (not shown) for removal of the pressurized liquid alkene (e.g., C₄ alkene) stream 17 and a fifth outlet (not shown) for removal of the alkene (e.g., C₂-C₄ alkene) vent gas stream 21. When needed, e.g., to empty and/or depressurize the condensate tank 13, a valve 23 may be opened to remove the alkene (e.g., C₂-C₄ alkene) vent gas stream 21, which optionally may be recycled to the condenser vessel 3.

At least one vaporizer 25 as described herein may also be present in the system 1, which may be operated under suitable conditions to produce pressurized alkene (e.g., C₂-C₄ alkene) gas stream 26 as described herein. The at least one vaporizer 25 may comprise a fourth inlet (not shown) for providing the pressurized liquid alkene (e.g., C₂-C₄ alkene) stream 17 and a sixth outlet (not shown) for removal of the pressurized alkene (e.g., C₂-C₄ alkene) gas stream 26. Advantageously, the pressurized alkene (e.g., C₂-C₄ alkene) gas stream 2.6 may have a pressure of about 100 kPa to about 2000 kPa and/or a temperature higher than a temperature of the pressurized liquid alkene (e.g., C₂-C₄ alkene) stream 17. The pressurized liquid alkene (e.g., C₂-C₄ alkene) stream 17 may be passed through the vaporizer 25 as determined by desired process conditions. For example, the pressurized liquid alkene (e.g., C₂-C₄ alkene) stream 17 may be passed through the vaporizer 25 when the pressure in the headspace of the at least condensate tank 13 reaches a predetermined value, e.g., a pressure of about 200 kPa to about 1500 kPa, as determined by a pressure sensor 22, where valve 19 may be closed and valve 24 may be opened. The system 1 may further comprise a valve 27 to maintain pressure within the system. Passing of the pressurized liquid alkene (e.g., C₂-C₄ alkene) stream 17 through the vaporizer 25 may continue for as long as determined by desired process conditions. Additionally or alternatively, passing of the pressurized liquid alkene (e.g., C₂-C₄ alkene) stream 17 through the vaporizer 25 may be halted when the pressurized liquid alkene (e.g., C₂-C₄ alkene) reaches a first low level as described herein as determined by a sensor 18 in the at least one condensate tank 13.

Optionally, if liquid alkenes (e.g., C₂-C₄ alkene) are desired, the system may also comprise a valve 28, which may be opened while valve 24 is closed, to transport the pressurized liquid alkene (e.g., C₂-C₄ alkene) stream 17.

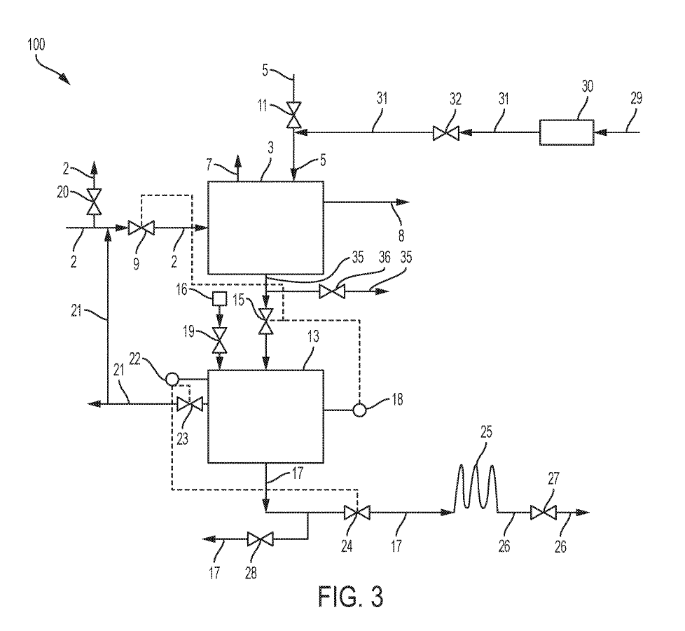

Optionally, as shown in FIG. 3, a system 100 may further comprise a gaseous nitrogen stream 29 and a heater 30 for producing a heated gaseous nitrogen stream 31, which may be introduced into the condenser vessel 3 as controlled by a valve 32, for example, during a defrost cycle as described herein. During the defrost cycle, valves 9, 11, and 15 may be closed, and the condenser vessel 3 may be heated via the gaseous nitrogen stream 29 to produce a second condensate stream 35 comprising alkenes (e.g., C₂-C₄ alkene). As shown in FIG. 3, the second condenstate stream 35 may be collected in the condensate tank 13 as controlled by valve 15 or the second condensate stream 35 may be transported to a different tank (not shown) as controlled by valve 36.

It is contemplated herein that the valves, streams, sensors, etc., shown in FIGS. 2 and 3 are not limited to the locations as shown in FIGS. 2 and 3, but may be present in the systems, as desired and needed by the requirements of the process. For example, valve 15 may be present on any suitable location on the first condensate stream 4 including near or at the third outlet (not shown) of the condenser vessel 3 or near or at the third inlet (not shown) of the at least one condensate tank 13.

### IV. Further Embodiments

The invention can additionally or alternatively include one or more of the following embodiments.

Embodiment 1. A method for producing pressurized C₂-C₄ alkene (e.g., ethylene and/or propylene) gas from a process gas stream comprising: a condensation cycle comprising: cooling the process gas stream comprising less than 80 wt % C₂-C₄ alkene (e.g., ethylene and/or propylene) in at least one condenser vessel with a cooling medium (e.g., liquid and/or gaseous nitrogen) under suitable conditions to produce a first condensate comprising C₂-C₄ alkene (e.g., ethylene and/or propylene) and a vent gas stream (e.g., comprising nitrogen), wherein the first condensate is collected in at least one condensate tank; and a pressurization cycle comprising: halting flow of the process gas stream and the cooling medium to the at least one condenser vessel and flow of the first condensate stream to the at least one condensate tank when the first condensate reaches a first high level in the at least one condensate tank; heating the at least one condensate tank to produce pressurized liquid C₂-C₄ alkene (e.g., ethylene and/or propylene) and a C₂-C₄ alkene (e.g., ethylene and/or propylene) vent gas; and optionally, passing the pressurized liquid C₂-C₄ alkene (e.g., ethylene and/or propylene) through a vaporizer operated under suitable conditions to produce the pressurized C₂-C₄ alkene (e.g., ethylene and/or propylene) gas having a pressure of at least about 100 kPa and a temperature higher than a temperature of the pressurized liquid C₂-C₄ alkene (e.g., ethylene and/or propylene).

Embodiment 2. The method of embodiment 1, wherein the process gas stream enters the at least one condenser vessel at a temperature of about −40° C. or lower and a pressure of at least about 90 kPa.

Embodiment 3. The method of embodiment 1 or 2, wherein the at least one condenser vessel comprises a shell and tube heat exchanger or a plate heat exchanger.

Embodiment 4. The method of any one of the previous embodiments, wherein the cooling medium is provided at a temperature of at least about −196° C. and a pressure of less than or equal to about 1500 kPa.

Embodiment 5. The method of any one of the previous embodiments, wherein passing the pressurized liquid C₂-C₄ alkene through the vaporizer is halted when the pressurized liquid C₂-C₄ alkene reaches a first low level in the at least one condensate tank.

Embodiment 6. The method of any one of the previous embodiments further comprising removing the C₂-C₄ alkene vent gas from the at least one condensate tank.

Embodiment 7. The method of any one of the previous embodiments further comprising a defrost cycle comprising: halting the process gas stream and the cooling medium to the at least one condenser vessel; heating the at least one condenser vessel to produce a second condensate stream comprising C₂-C₄ alkene; and collecting the second condensate stream in a second condensate tank.

Embodiment 8. The method of embodiment 7, wherein the heating of the at least one condenser vessel is provided by gaseous nitrogen.

Embodiment 9. The method of embodiment 8 further comprising heating the gaseous nitrogen.

Embodiment 10. The method of any one of the previous embodiments, wherein the condensation cycle, the pressurization cycle, and the defrost cycle are running in parallel in two or more condenser vessels.

Embodiment 11. The method of any one of the previous embodiments, wherein the condensation cycle runs for about 6.0 hours to about 24 hours, the pressurization cycle runs for about 6.0 to about 24 hours and/or the defrost cycle runs for about 1.0 hour to about 3.0 hours.

Embodiment 12. A system for producing a pressurized C₂-C₄ alkene (e.g., ethylene and/or propylene) gas from a process gas stream comprising: a process gas stream comprising less than 80 wt % C₂-C₄ alkene (e.g., ethylene and/or propylene); a cooling medium (e.g., liquid or gaseous nitrogen) stream; a vent gas stream; a first condensate stream comprising C₂-C₄ alkene (e.g., ethylene and/or propylene); a pressurized liquid C₄ alkene (e.g., ethylene and/or propylene) stream; a C₂-C₄ alkene (e.g., ethylene and/or propylene) vent gas stream; a pressurized C₂-C₄ alkene (e.g., ethylene and/or propylene) gas stream; at least one condenser vessel operated under suitable conditions to produce the first condensate stream comprising C₂-C₄ alkene (e.g., ethylene and/or propylene) and the vent gas stream, wherein the at least one condenser vessel comprises: a shell and tube heat exchanger or a plate heat exchanger; a first inlet for providing the process gas stream; a second inlet for providing the cooling medium; a first outlet for removal of a spent cooling medium; a second outlet for removal of the vent gas stream; and a third outlet for removal of the first condensate stream; at least one condensate tank, wherein the at least one condensate tank comprises: a third inlet for providing the first condensate; a fourth outlet for removal of the pressurized liquid C₂-C₄ alkene (e.g., ethylene and/or propylene) stream; and a fifth outlet for removal the C₂-C₄ alkene (e.g., ethylene and/or propylene) vent gas stream; a means for providing heat to the at least one condensate tank; and at least one vaporizer operated under suitable conditions to produce the pressurized C₂-C₄ alkene (e.g., ethylene and/or propylene) gas stream having a pressure of at least about 100 kPa and a temperature higher than a temperature of the pressurized liquid C₂-C₄ alkene (e.g., ethylene and/or propylene), wherein the vaporizer comprises: a fourth inlet for providing the pressurized liquid C₂-C₄ alkene (e.g., ethylene and/or propylene) stream; and a sixth outlet for removal of the pressurized C₂-C₄ alkene (e.g., ethylene and/or propylene) gas stream.

Embodiment 13. The system of embodiment 12, wherein the cooling medium is provided at a temperature of at least about −196° C. and a pressure of less than or equal to about 1500 kPa.

Embodiment 14. The system of embodiment 12 or 13, wherein the C₂-C₄ alkene is ethylene and/or propylene.

What is claimed is:

1. A method for producing pressurized C₂-C₄ alkene gas from a process gas stream comprising:
a condensation cycle comprising:
cooling the process gas stream comprising less than 80 wt % C₂-C₄ alkene in at least one condenser vessel with a cooling medium under suitable conditions to produce a first condensate comprising C₂-C₄ alkene and a vent gas stream, wherein the first condensate is collected in at least one condensate tank; and
a pressurization cycle comprising:
halting flow of the process gas stream and the cooling medium to the at least one condenser vessel and flow of the first condensate stream to the at least one condensate tank when the first condensate reaches a first high level in the at least one condensate tank; and
heating the at least one condensate tank to produce pressurized liquid C₂-C₄ alkene and a C₂-C₄ alkene vent gas.

2. The method of claim 1, wherein the process gas stream enters the at least one condenser vessel at a temperature of about −40° C. or lower and a pressure of at least about 90 kPa.

3. The method of claim 1, wherein the vent gas stream comprises nitrogen.

4. The method of claim 1, wherein the cooling medium is liquid nitrogen and/or gaseous nitrogen.

5. The method of claim 1, wherein the cooling medium is provided at a temperature of at least about −196° C. and a pressure of less than or equal to about 1500 kPa.

6. The method of claim 1, wherein the at least one condenser vessel comprises a shell and tube heat exchanger or a plate heat exchanger.

7. The method of claim 1 further comprising passing the pressurized liquid C₂-C₄ alkene through a vaporizer operated under suitable conditions to produce the pressurized C₂-C₄ alkene gas having a pressure of at least about 100 kPa and a temperature higher than a temperature of the pressurized liquid C₂-C₄ alkene.

8. The method of claim 7, wherein passing the pressurized liquid C₂-C₄ alkene through the vaporizer is halted when the first condensate reaches a first low level in the at least one condensate tank.

9. The method of claim 1 further comprising removing the C₂-C₄ alkene vent gas from the at least one condensate tank.

10. The method of claim 1 further comprising a defrost cycle comprising:
halting the process gas stream and the cooling medium to the at least one condenser vessel;
heating the at least one condenser vessel to produce a second condensate stream comprising C₂-C₄ alkene; and
collecting the second condensate stream a second condensate tank.

11. The method of claim 10, wherein the heating of the at least one condenser vessel is provided by gaseous nitrogen.

12. The method of claim 11 further comprising heating the gaseous nitrogen.

13. The method of claim 10, wherein the condensation cycle runs for about 6.0 hours to about 24 hours, the pressurization cycle runs for about 6.0 to about 24 hours and the defrost cycle runs for about 1.0 hour to about 3.0 hours.

14. The method of claim 10, wherein the condensation cycle, the pressurization cycle, and the defrost cycle are running in parallel in two or more condenser vessels.

15. The method of claim 1, wherein the C₂-C₄ alkene is ethylene and/or propylene.

16. A system for producing a pressurized C₂-C₄ alkene gas from a process gas stream comprising:
a process gas stream comprising less than 80 wt % C₂-C₄ alkene;
a cooling medium stream;
a vent gas stream;
a first condensate stream comprising C₂-C₄ alkene;
a pressurized liquid C₂-C₄ alkene stream;
a C₂-C₄ alkene vent gas stream;
a pressurized C₂-C₄ alkene gas stream;
at least one condenser vessel operated under suitable conditions to produce the first condensate stream comprising C₂-C₄ alkene and the vent gas stream, and wherein the at least one condenser vessel comprises:
a shell and tube heat exchanger or a plate heat exchanger;
a first inlet for providing the process gas stream;
a second inlet for providing the cooling medium;
a first outlet for removal of a spent cooling medium;
a second outlet for removal of the vent gas stream; and
a third outlet for removal of the first condensate stream;
at least one condensate tank, wherein the at least one condensate tank comprises:
a third inlet for providing the first condensate stream;
a fourth outlet for removal of the pressurized liquid C₂-C₄ alkene stream; and
a fifth outlet for removal the C₂-C₄ alkene vent gas stream;
a means for providing heat to the at least one condensate tank; and
at least one vaporizer operated under suitable conditions to produce the pressurized C₂-C₄ alkene gas stream having a pressure of at least about 100 kPa and a temperature higher than a temperature of the pressurized liquid C₂-C₄ alkene, wherein the vaporizer comprises:
a fourth inlet for providing the pressurized liquid C₂-C₄ alkene stream; and
a sixth outlet for removal of the pressurized C₂-C₄ alkene gas stream.

17. The system of claim 16, wherein the cooling medium is liquid nitrogen and/or gaseous nitrogen.

18. The system of claim 16, wherein the cooling medium is provided at a temperature of at least about −196° C. and a pressure of less than or equal to about 1500 kPa

19. The system of claim 16, wherein the C₂-C₄ alkene is ethylene and/or propylene.
